SEQUENCE LISTING (4 sequences)

SEQ ID NO: 1 - DNA, 405 bp, Homo sapiens
  allele (205): G 205 T
taattatgggtctgtaaccaccctggactgggtgctcctcactgacggacttgtctgaacctctctttgtctccagcgcccagcactgggcctggcaaaacctgagacgcccggtacatgttggccaaatgaatgaaccagattcagaccggcaggggcgctgtggtttaggaggggcctggggtttctcccaggaggtttttgkgcttgcgctggagggctctggactcccgtttgcgccagtggcctgcatcctggtcctgtcttcctcatgtttgaatttctttgctttcctagtctggggagcagggaggagccctgtgccctgtcccaggatccatgggtaggaacaccatggacagggagagcaaacggggccatctgtcaccaggggcttagggaagg

SEQ ID NO: 2 - DNA, 405 bp, Homo sapiens
  allele (233): G 233 A
taattatgggtctgtaaccaccctggactgggtgctcctcactgacggacttgtctgaacctctctttgtctccagcgcccagcactgggcctggcaaaacctgagacgcccggtacatgttggccaaatgaatgaaccagattcagaccggcaggggcgctgtggtttaggaggggcctggggtttctcccaggaggtttttgggcttgcgctggagggctctggactcccrtttgcgccagtggcctgcatcctggtcctgtcttcctcatgtttgaatttctttgctttcctagtctggggagcagggaggagccctgtgccctgtcccaggatccatgggtaggaacaccatggacagggagagcaaacggggccatctgtcaccaggggcttagggaagg

SEQ ID NO: 3 - DNA, 277 bp, Homo sapiens
  allele (121): C 8166 T
cagtgagtcccgttcgtccttcaccagtggctgggcccctggctgcctctggctgcagaggggtgtctggaggacgcacggtgggtggacccgctcctgccttgggccagacttcaacgayaggtccagggcctggtctgactcttctgggacctggcagggaggaggcccagatgctcgaggagggagggcagccttgaccccaaacggggggagcttggcctttcgggcagctgggcagaggtccgcggtccagacaggccaggggcacggtggc

SEQ ID NO: 4 - DNA, 277 bp, Homo sapiens
  allele (140): C 11898 A
atgcgccctaggggacagcagaccatcctcctttcctgcatggttagcatagcaacagcctcctcctctgggttgagaaggcctgggctgagtcatgggcccaactgggaacatgagagggcctccctgcaggacagggmactggtccaaactcagcaggaggcacggtgggtgtgggagggggtaggggaggagccagcaccacctcccagaaggaaggacctgcatttggggtcaggactgggaaggcggcaggagctgacccaagaggggcagg

(19) United States
(12) Patent Application Publication
(10) Pub. No.: US 2007/0207468 A1
(43) Pub. Date: Sep. 6, 2007

(54) 12 kb sequence immediately adjacent to and upstream of the AKT1 gene locus containing multiple genetic variations associated with changes in metabolic syndrome, and methods of use

(76) Inventors: Eric P. Hoffman, Kensington, MD (US); Joseph M. Devaney, Silver Spring, MD (US); Heather Gordish-Dressman, Baltimore, MD (US)

Correspondence Address: DR. MELVIN BLECHER, 4329 VAN NESS ST., NW, WASHINGTON, DC, 20016, US

(21) Appl. No.: 11/367,481

(22) Filed: Mar. 6, 2006

(51) Int. Cl. C12Q 1/68; C07H 21/04

(52) U.S. Cl. 435/6; 536/23.2

(57) ABSTRACT

We have identified a 12 kb sequence immediately upstream of the AKT1 gene locus in humans that contains single nucleotide polymorphic polynucleotide (SNPs) whose alleles in strong linkage disequilibrium, and that are prognostic for metabolic syndrome and its metabolic sequellae such as hyperglycemia and type 2 diabetes. The +G205T allele is particularly highly predictive of the potential for protection against metabolic syndrome and its sequellae. The detection of these SNPs by the techniques described herein forms the foundation for methods for genotype-specific clinical interventions designed to slow the rapid population increases in metabolic syndrome and its metabolic sequellae.

Acknowledgements: Supported in part by NIH grant RO1 NS40606 (co-support by NINDS, NIAMS, and NIA) and NO1-AG-6-2101, -2103 and -2106, Principal Investigator, Dr. E. P. Hoffman. The United States Government may, therefore, have an interest in this invention.

### FIELD OF THE INVENTION

The invention is generally related to defining and identifying quantitative trait loci (QTSs) that contribute to abnormal metabolism in humans. More specifically, the invention relates to SNP sequences derived from a 12 kb sequence immediately adjacent upstream to the AKT1 gene locus that contains alleles that are in linkage disequilibrium and are associated with the the potential for metabolic syndrome and its metabolic sequellae, and methods for the use of these SNPs for prognostic and clinical intervention purposes.

### BACKGROUND

The incidence of obesity, metabolic syndrome, hyperglycemia, and type 2 diabetes is an epidemic in most industrialized populations. Obesity, as defied by body mass index (SMI>30), has risen in the USA from 10% of women in 1990 to 20% of women in 2002, with some states reporting >25% obesity rates. African-American and Hispanic populations are at significantly higher risk, and it has been calculated that a Hispanic child born in 2000 will have about a 50% risk of developing type 2 diabetes in his or her lifetime, with an associated loss of 18-22 quality-adjusted life years.

We have previously identified and isolated a 12 kb sequence immediately upstream of the AKT1 gene locus that contains eight single nucleotide polymorphic polynucleotides (SNPs) in 4 haplotype regions, the loci in each haplotype being in linkage disequilibrium, that show association with body composition, Body Mass Index, muscle mass, obesity and AKT1 expression enhancement in human males (Hoffman, EP et al., U.S. patent application Ser. No. 11/635,377, incorporated herein by reference). A four QTL haplotype (Haplotype2) was defined where residues within highly conserved regulatory regions were altered. This haplotype explained up to 26% of population variation in bone cortical volume, 12% of subcutaneous fat volume, and 9% of strength variation, resulting in a body build with large bones, strong muscles, and low subcutaneous fat. Other SNPs detect, presymptomatically, the potential for increased amounts of subcutaneous fat. The detection of these SNPs form the foundation for genotype-specific clinical interventions designed to slow the rapid population increased in obesity.

The dramatic rise in obesity in the United States has lead to an equally alarming increase in the percentage of the population who suffer from metabolic syndrome and its sequellae. Metabolic syndrome is a clustering of atherosclerotic cardiovascular disease risk factors, such as hypertension, dyslipidemia, insulin resistance with glucose intolerance (type 2 diabetes), low levels of HDL, a systemic proinflammatory state, impaired fibrinolysis, procoagulation and, most telling, central obesity. It is generally acknowledged that insulin resistance is the primary cause of hyperglycemia with type 2 diabetes, and that it is closely correlated with visceral adiposity (obesity). Type 2 diabetes is characterized by persistent hyperglycemia in the face of adequate amounts of circulating insulin and a functional pancreas; hence, it also referred to as insulin-independent diabetes. Because it generally occurs in individuals over the age of 40, it also has been referred to as late-onset diabetes.

However, it is also generally recognized that obesity alone does not always result in the metabolic syndrome, and that genetics plays an important role. Indeed, about 75% of metabolic syndrome cases arise from genetic factors, and that only 25% of the cases are due to the environment. There are genetic propensities to become obese and insulin resistant, independent of ethnicity, extent of inactivity and food intake. This genetic predisposition extends through all populations, with BMI, as one harbinger of obesity and metabolic syndrome, thought to show 75% heritability (Perusse, L et al. Obes Res 8:26 (2000)).

Because of this close genetic association between obesity and the symptoms of metabolic syndrome, and because of the obvious value in identifying genetic loci that are prognostic for metabolic syndrome prior to the onset of the manifestations of the syndrome, we sought and found QTLs in linkage disequilibrium that are associated with metabolic syndrome within the same 12 kb polynucleotide DNA sequence immediately upstream of the AKT1 gene locus in which we had also found QTLs for elements of body composition, including muscle mass, bone density and adiposity and BMI. The identity of these QTLs and their use in predicting the onset of anatomical and metabolic changes in humans are described below.

### SUMMARY OF THE INVENTION

We have identified a 12 kb sequence region immediately upstream of the AKT1 gene locus in humans, the 12 kb sequence including the first exon and upstream regulatory regions that contains a haplotype (Haplotype 2) consisting of single nucleotide polymorphic polynucleotides (“SNPs”) whose alleles are in strong linkage disequilibrium with each other, and that are associated with the potential for metabolic syndrome and its metabolic sequellae. The sequences of Haplotype 2 containing four of these SNPs are shown below as SEQ ID Nos. 1 through 4, or complementary strands thereof.

In one embodiment the alleles of the four aforementioned SNPs are, respectively +G205T (SEQ. ID No. 1), +G233A (SEQ ID No. 2), −C8166T (SEQ. ID No.3) and —C11898A, (SEQ. ID No. 4).

In another embodiment, the alleles are associated with increased protection against metabolic syndrome and a decreased potential for accompanying hyperglycemia and type 2 diabetes.

In yet another embodiment, the allele +G205T is strongly associated with a protective effect against metabolic syndrome and a decreased potential for Type 2 diabetes.

In still another embodiment, the alleles are used in a prognostic method for the potential for metabolic syndrome and its metabolic sequellae.

In another embodiment, the alleles are used in establishing the need for clinical interventions in the treatment presymptomatically of metabolic syndrome and its metabolic sequellae.

### DETAILED DESCRIPTION OF THE PREFERRED EMBODIMENTS

We have identified a 12 kb DNA sequence immediately upstream of the AKT1 gene locus, and have discovered within this 12 kb DNA sequence a series of haplotypes, each containing one or more SNPs, each with a single allele that is in strong linkage disequilibrium with the other alleles in the 12 kb sequence, each SNP being associated with elements of body composition and the potential for presenting with metabolic syndrome and its sequellae in humans subjects in the FAMuSS young adult cohort (Hoffman EP et al., U.S. patent application Ser. No. 11/635,377, incorporated herein by reference) and the Health ABC older adult cohort (see details below). The elements of body composition associated with these QTRL are increased muscle strength, bone size, and decreased subcutaneous fat in males, and the suggestion of association with BMI in both sexes.

The present discoveries are based on data obtained from the 3,001 White and Black volunteer Health ABC study population, consisting of older individuals from Memphis and Pittsburgh (Table 1) (Goodpaster, B H et al, J Appl Physiol 90:2157 (2001)).

Metabolic Syndrome can be defined by fulfilling standard criteria (see below for the criteria employed in this study), and data analyzed for the entire population, and then stratified by sex and ethnicity. In our population, we discovered that the alleles in the four SNPs in Haplotype 2 of the aforementioned 12 kb sequence were protective against metabolic syndrome, with homozygotes showing about half the incidence of metabolic syndrome in both the entire non-stratified cohort (odds ratio 0.6 [95% Cl 0.457-0.788]; p<0.001), and by most stratifications by sex and ethnicity (Table 2). The associations were strongest in Black males.

Logistic regression was used to test the association between AKT1 and metabolic syndrome. All models were adjusted for age and body weight. Associations between fasting glucose metabolic syndrome criteria and AKT1 genotype (Table 3) were assessed using Chi square tests. Assessment of the predictive ability of AKT1 and other metabolic syndrome criteria used generalized liner regression models and likelihood ratio tests to compare models containing the predictor, age and body weight to models containing age and body weight only.

We found that the diagnosis of metabolic syndrome was dependent on five criteria; glycemic index (insulin resistance) blood pressure, HDL levels, triglyceride levels, and waist circumference (adiposity). Patients who showed abnormal threshold levels of three of the five criteria were typically given the diagnosis of “metabolic syndrome.” We studied the relationship of AKT1 haplotypes with each of these five component measures, and found that fasting glucose levels in males was most strongly associated with AKT1 genotype. Using a dominant inheritance model, “all males” and Black males with the alleles identified herein showed significantly decreased fasting glucose levels (Chi²=8.3; p=0.01). This suggests that AKT1 plays a regulatory role in maintenance of glucose levels in the blood, perhaps via insulin resistance.

Measures of hyperglycemia and increased insulin resistance are generally acknowledged to be physiologically related, and are considered key components of metabolic syndrome and type 2 diabetes. The best measures of these parameters remain debated; while fasting glucose is most commonly used, HOMA (fasting glucose×insulin), oral glucose tolerance test, and intravenous glucose tolerance test are often employed as well. We had both fasting glucose and HOMA available in our Health ABC population, and we tested the predictive power of these measures for metabolic syndrome diagnosis, as well as systolic blood pressure, and HDL levels (Table 2). Correlation coefficients (R²) without statistical adjustment for covariance showed HDL levels to have the highest predictive power, followed by fasting glucose, HOMA, AKT1 genotype, and systolic blood pressure (Table 3). However, all standard criteria for metabolic syndrome showed strong co-variance with age and weight. We therefore statistically adjusted for age and weight, and re-tested correlations. HDS and fasting glucose remained the most highly predictive; however, AKT1 genotype became similar in predictive power to HOMA and blood pressure (Table 3).

AKT1 is also increasingly recognized for its role in metabolism and insulin signaling. Although AKT2 is more highly expressed in insulin-sensitive tissues, both AKT1 and AKT2 are downstream of the key phosphatidylinositol 3-kinase pathway (Pl3k). The Pl3k pathway is critical for a cell's response to leptin, regulation of the insulin receptor, response to IGF-1, and many other signaling pathways. AKT1 is involved in intramuscular insulin signaling and has also been linked to muscle hypertrophy and angiogenic growth factor synthesis.

The easy availability of relatively inexpensive, high calorie food, coupled with low physical activity levels are driving the rapid rise in obesity and type 2 diabetes. Indeed, poor diet and low physical activity are expected to overcome tobacco as the single most common cause of premature death. However, it is also clear that individuals show different propensities to become obese, with certain genetically derived “physiotypes” that help set lean body mass (muscle content), bone size, and fat deposits. An individual's genetically-determined tendency to become obese or remain lean seems conserved throughout primates. For example, in carefully controlled studies of rhesus monkey populations that were provided unlimited food, only a subset of individuals become morbidly obese. Studies of the progression from obesity to type 2 diabetes have shown that muscle insulin resistance is one of the earlier stages of this process. Thus, fat tissue and muscle show endocrine actions that work together with the pancreatic beta cells and liver to regulate energy balance throughout the body, and each of these tissues contribute to a specific energy balance “set point” specific to each individual (genetically determined), yet influenced by environment. The identification of genetic risk factors for obesity-related physiotypes could be considered a key first step in developing personalized interventions to prevent obesity and the associated morbidity factors.

Given the strong predictive correlations between BMI, subcutaneous fat, and risk for subsequent obesity, metabolic syndrome and its sequellae such as hyperglycemia and type 2 diabetes, our discoveries suggest that genetic testing of the four alleles discovered here will, together with the alleles previously discovered (Hoffman et al., above), identify individuals at lower risk for poor muscle strength, weak bones, obesity, metabolic syndrome and its sequellae, including type 2 diabetes.

### EXAMPLES

### Example 1

### Method of Isolation and Identification of the SNPs and their Alleles

In the methods of the invention, details of which are to be found in U.S. patent application Ser. No. 11/635377 (incorporated herein by reference), a patient's genomic DNA was extracted from blood or buccal cells using the PUREGENE DNA Purification System (Gentra Systems, Minneapolis, Minn.) according to the manufacturer's instructions. Briefly, for whole blood the extraction process was as follows: (1) Red blood cells were lysed and the contents of the red blood cells removed; (2) nucleated cells were lysed, thus exposing proteins and DNA; (3) proteins were precipitated and removed; and (4) DNA was isolated by alcohol precipitation and placed in a DNA hydration solution. DNA was extracted from nucleated cells as follows: (1) cells were lysed, thereby liberating DNA and proteins; (2) proteins were precipitated and removed; and, (3) DNA was isolated using alcohol precipitation, and placed in a DNA hydration solution.

This DNA was analyzed for the presence of one or more of the 4 allelic nucleotides of SEQ ID Nos. 1 through 4, or their complementary strands, by standard methods well known in this art. The pattern of alleles and haplotypes will thereby predict which clinical intervention is best suited for the patient in order to decrease the risk of metabolic syndrome and sequellae such as hyperglycemia, glucose resistance and type 2 diabetes.

### Implications of AKT1 Genotypes and Human Evolution

The easy availability of relatively inexpensive, high calorie food, coupled with low physical activity levels are driving the rapid rise in obesity and type 2 diabetes. Indeed, poor diet and low physical activity are expected to overcome tobacco as the single most common cause of premature death. ¹However, it is also clear that individuals show different propensities to becoming obese, with certain genetically derived “physiotypes” that help set lean body mass (muscle content), bone size, and fat deposits. The data presented here and in the pending patent application discussed above shows that AKT1 genotype is a major physiotype marker in human populations, with young men showing stronger muscles, larger bones, and less subcutaneous fat with certain alleles. We have now found that alleles from the 12 kb region protect against metabolic syndrome in both men and women. The protective effect in men appears to be linked to decreased fasting glucose levels, suggesting that AKT1 protein plays a role in glycemic control. Our data suggests that pre-symptomatic testing for AKT1 genotypes, particularly when using the +G205T locus as a key haplotype marker, will help identify individuals at risk for metabolic syndrome. Specific clinical interventions that may be optimally targeted towards persons of specific AKT1 genotypes can be devised based on the invention herein. For example, our data suggests AKT1 genotype can identify individuals that may be predisposed to problems with glycemic control, and dietary or drug interventions employed presymptomatically.

TABLE 1
Demographics of the Health ABC study population.

| | Males | Females |
|---|---|---|
| Ethnicity | | |
| White | 926 (63.5%) | 836 (54.2%) |
| Black | 533 (36.5%) | 706 (45.8%) |
| Total | 1459 | 1542 |
| Collection site | | |
| Memphis | 733 (50.2%) | 764 (49.5%) |
| Pittsburgh | 726 (49.8%) | 778 (50.4%) |
| Total | 1459 | 1542 |
| Age (years) | 73.75 ± 2.87 | 73.49 ± 2.88 |
| Baseline BMI | 27.09 ± 3.96 | 27.74 ± 5.50 |
| Baseline body weight | 81.47 ± 13.30 | 70.62 ± 14.71 |
| (kg) | | |

TABLE 2
AKTI haplotype 2 is protective of metabolic syndrome
in the Health ABC cohort.

| | AKTI | | N | | | |
|---|---|---|---|---|---|---|
| | (+G205T) | N | (metabolic | Odds | | 95% Confidence |
| Group | Genotype | (controls) | syndrome) * | ratio | P-value | Interval |
| All subjects | GG | 812 | 566 | 1.00 | | |
| | GT | 823 | 490 | 0.806 | 0.010 | 0.685–0.948 |
| | TT | 205 | 86 | 0.604 | <0.001 | 0.457–0.798 |
| All males | GG | 408 | 251 | 1.00 | | |
| | GT | 477 | 202 | 0.662 | 0.001 | 0.515–0.852 |
| | TT | 105 | 42 | 0.598 | 0.018 | 0.391–0.915 |
| White males | GG | 275 | 179 | 1.00 | | |
| | GT | 247 | 232 | 0.764 | 0.087 | 0.562–1.039 |
| | TT | 53 | 26 | 0.674 | 0.161 | 0.389–1.169 |
| Black males | GG | 133 | 72 | 1.00 | | |
| | GT | 200 | 59 | 0.539 | 0.007 | 0.343–0.846 |
| | TT | 52 | 16 | 0.567 | 0.106 | 0.285–1.128 |
| All females | GG | 404 | 325 | | | |
| | GT | 376 | 288 | 0.932 | 0.540 | 0.744–1.167 |
| | TT | 100 | 54 | 0.577 | 0.005 | 0.391–0.849 |
| White | GG | 236 | 186 | 1.00 | | |
| females | GT | 205 | 149 | 0.852 | 0.307 | 0.626–1.158 |
| | TT | 39 | 18 | 0.541 | 0.061 | 0.284–1.029 |
| Black | GG | 168 | 129 | 1.00 | | |
| females | GT | 201 | 139 | 1.07 | 0.673 | 0.766–1.509 |
| | TT | 61 | 36 | 0.721 | 0.197 | 0.438–1.184 |

TABLE 3
AKTI genotype is a sensitive predictor of metabolic syndrome
when adjusted for age and body weight.

| | R² with | R² with | Chi² and |
|---|---|---|---|
| | no other | age and | p-value from |
| Predictor | predictors | body weight | Likelihood ratio test |
| AKTI | 0.0113 | 0.1550 | 5.94; 0.015 |
| Fasting glucose | 0.0968 | 0.2274 | 51.79; <0.001 |
| HOMA* | 0.0535 | 0.1389 | 6.73; 0.013 |
| Systolic blood | 0.0044 | 0.1506 | 2.59; NS |
| pressure | | | |
| HDL | 0.1778 | 0.2697 | 75.93; <0.001 |

*(HOMA (IR) = [(glucose in mg/dl * 0.05551) * (insulin in microIU/ml)]/22.5)

What is claimed is:

1. A composition, comprising a single polymorphic polynucleotide (SNP) located in the 12 kb DNA sequence immediately upstream of the AKT1 gene locus in humans, said 12 kb sequence including the first exon and upstream regulatory regions, and said SNP being a member of a haplotype whose alleles are in linkage disequilibrium, wherein said SNP is associated with protection against metabolic syndrome and its metabolic sequellae.

2. The composition of claim 1 wherein said SNP is selected from the haplotype group consisting of SEQ ID Nos. 1 through 4, or a complementary strand of SEQ ID Nos. 1 through 4

3. The composition according to claim 2, wherein said SNP has the sequence shown in SEQ ID No. 1, and said allele is +G205T.

4. The composition according to claim 2, wherein said SNP has the sequence shown in SEQ ID No. 2 and said allele is +G233A.

5. The composition according to claim 2, wherein said SNP has the sequence shown in SEQ ID No. 3, and said allele is −C8,166T

6. The composition according to claim 2, wherein said SNP has the sequence shown in SEQ ID No. 4, and said allele is −C11,898A.

7. A method for predicting presymptomatically the likelihood that a human will have a genetic propensity for metabolic syndrome and its metabolic sequellae, comprising the steps of obtaining a tissue sample from said human, isolating genomic DNA from said tissue sample, assaying said genomic DNA for the presence or absence of the SNP of claim 1, wherein the presence of said SNP in said DNA sample predicts protection against said metabolic syndrome and its metabolic sequellae.

8. The method according to claim 7, wherein said SNP has the sequence of SEQ ID No. 1, and said allele is +G205T.

9. The method according to claim 7, wherein said SNP has the sequence of SEQ ID No 2, and said allele is +G233A.

10. The method according to claim 7, wherein said SNP has the sequence of SEQ ID No. 3, and said allele is −C8,166T.

11. The method according to claim 7, wherein said SNP has the sequence of SEQ ID No. 4, and said allele is −C11,898A.

12. A method of clinical intervention against metabolic syndrome and its metabolic sequellae in a human, comprising the steps of (i) assaying for the presence or absence of the SNP of claim 1 in the genomic DNA of said human; and (ii) administering a clinical regimen, where needed, appropriate to the presence or absence of a protective allele in said human and the potential for the onset of the metabolic syndrome and its metabolic sequellae.

13. The method according to claim 12, wherein said SNP has the sequence of SEQ ID No. 1, and said allele is +G205T.

14. The method according to claim 12, wherein said SNP has the sequence of SEQ ID No. 2, and said allele is −G233A.

15. The method according to claim 12 wherein said SNP has the sequence of SEQ ID No. 3, and said allele is −C8,166T.

16. The method according to claim 12, wherein said SNP has the sequence of SEQ ID No. 4, and said allele is −C11,898A.
